(19) United States
(12) Patent Application Publication
(10) Pub. No.: US 2023/0061132 A1
(43) Pub. Date: Mar. 2, 2023

(54) CELL CULTURE VESSEL

(71) Applicant: LG ELECTRONICS INC., Seoul (KR)

(72) Inventors: Sanggeun CHO, Seoul (KR); Bumjin BAE, Seoul (KR); Muntae JUNG, Seoul (KR)

(73) Assignee: LG ELECTRONICS INC., Seoul (KR)

(21) Appl. No.: 17/799,606

(22) PCT Filed: Feb. 13, 2020
(86) PCT No.: PCT/KR2020/002009

§ 371 Date: Aug. 12, 2022

(51) Int. Cl. C12M 1/42; C12M 1/00

(52) U.S. Cl. CPC C12M 1/42; C12M 35/02; C12M 23/20

(57) ABSTRACT

A cell culture vessel according to an embodiment of the present invention comprises: a base substrate; at least one deposited graphene layer provided on the base substrate; and a culture substrate comprising at least one electrostimulation input terminal coupled to a working electrode and configured to transmit electrical stimuli to the deposited graphene layer.

### BACKGROUND OF THE DISCLOSURE

### 1. Field of the Disclosure

The present disclosure relates to a cell culture vessel. More particularly, the present disclosure relates to a cell culture vessel capable of effectively applying electrical stimuli to a deposited graphene layer included in a culture substrate.

### 2. Description of the Related Art

Graphene is a structure in which hexagons of six carbon atoms are connected to each other to form a two-dimensional single layer, and has a structure different from zero-dimensional fullerene, carbon nanotube having a tube-shaped one-dimensional structure, and graphite having a three-dimensional structure.

In a conventional cell culture vessel, a substrate is subjected to corona treatment or coated with extracellular matrix (ECM), and culture and differentiation are performed without external stimuli, or culture and differentiation are performed by applying external stimuli such as an electric field.

Specifically, the substrate may be spaced apart from a working electrode and a counter electrode to be disposed between the working electrode and the counter electrode, and cells cultured on the substrate may get the electrical stimuli by the electric field generated between the working electrode and the counter electrode.

However, in this case, since the culture cells on the substrate indirectly gets the electrical stimuli by the electric field, the electrical stimuli may not be effectively transmitted to the culture cells and thereby culture efficiency may be deteriorated.

### SUMMARY

The technical problems to be solved by the present disclosure will be described as follows.

First, the present disclosure is to provide a cell culture vessel capable of directly applying electrical stimuli to a culture cell.

Further, the present disclosure is to solve all problems that may be caused or predicted from the prior art, in addition to the above-described technical problems.

A cell culture vessel according to the present disclosure includes a culture substrate including a base substrate, at least one deposited graphene layer disposed on the base substrate, and at least one electrostimulation input terminal coupled to a working electrode and configured to transmit electrical stimuli to the deposited graphene layer.

The electrostimulation input terminal may be directly coupled to the deposited graphene layer.

A plurality of electrostimulation input terminals may be disposed on an edge of the deposited graphene layer to be spaced apart from each other.

The base substrate may include polystyrene (PS).

The base substrate may include a conductive metal layer, and the conductive metal layer may include at least one of ITO, Ag, Au, or Cu.

The deposited graphene layer may be directly disposed on the base substrate, and the electrostimulation input terminal may be directly coupled to the base substrate.

The cell culture vessel may further include the working electrode transmitting electrical stimuli to the deposited graphene layer, and a counter electrode electrically opposite to the working electrode. The working electrode may be directly coupled to the culture substrate, and the counter electrode may be located to be spaced apart from the culture substrate.

Each of the working electrode and the counter electrode may have a plate shape parallel to the culture substrate or a linear shape intersecting the culture substrate.

The cell culture vessel may further include a culture substrate case including a case bottom on which the culture substrate is placed and an outer wall surrounding an edge of the case bottom, and a culture substrate case cover covering an upper opening of the culture substrate case. The culture substrate case cover may include at least one electrode insertion hole through which at least one of the working electrode or the counter electrode passes.

The working electrode and the counter electrode may be connected to a power supply.

At least one of the working electrode or the counter electrode may include at least one of Pt, Au, Ag, Ti, or stainless steel.

The deposited graphene layer may be a single layer or a multiple layer in which a plurality of layers are stacked, and the number of layers in the deposited graphene layer may be 1 to 5.

An oxygen functional group may be formed on a surface of the deposited graphene layer.

### Effects of the Disclosure

The effects of a cell culture vessel according to the present disclosure configured as described above are as follows.

The cell culture vessel of the present disclosure includes an input terminal which may transmit electrical stimuli to a deposited graphene layer, thus effectively transmitting the electrical stimuli to cultured cells and thereby improving culture efficiency.

### BRIEF DESCRIPTION OF THE DRAWINGS

FIG. 1 is a perspective view of a cell culture vessel according to an embodiment of the present disclosure.

FIG. 2 is an exploded perspective view of the cell culture vessel according to an embodiment of the present disclosure.

FIG. 3 is a plan view of a culture substrate according to an embodiment of the present disclosure.

FIG. 4 is a plan view of a culture substrate according to another embodiment of the present disclosure.

FIG. 5 shows cell culture results according to an example of the present disclosure and a comparative example.

FIG. 6 is a sectional view of the cell culture vessel according to an embodiment of the present disclosure.

### DETAILED DESCRIPTION OF THE PREFERRED EMBODIMENTS

Advantages and features of the present disclosure and a method of achieving them will become apparent with reference to the following embodiments.

The present disclosure will be defined by the scope of claims. If there is a separate description for the meaning of terms in the specification, the meaning of the terms will be defined by the above description. Like reference numerals refer to like elements throughout the specification.

A cell culture vessel 100 of the present disclosure may dispose a deposited graphene layer 12 on a base substrate 11, and may include an electrostimulation input terminal that may transmit electrical stimuli to the deposited graphene layer 12, thus effectively transmitting the electrical stimuli to a culture cell.

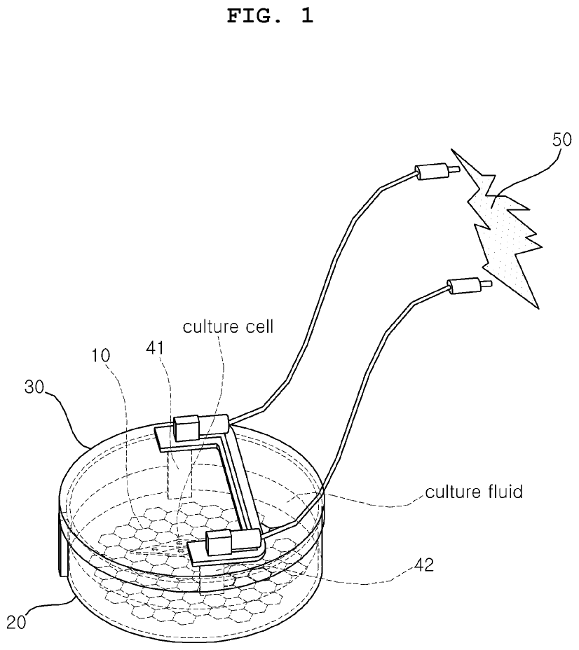

In this connection, the cell culture vessel 100 according to an embodiment of the present disclosure will be described with reference to FIGS. 1 to 6.

First, referring to FIGS. 1 to 3, the cell culture vessel 100 may include a culture substrate 10 in which a cell is directly cultured.

Specifically, the culture substrate 10 may include a base substrate 11, at least one deposited graphene layer 12 disposed on the base substrate 11, and at least one electrostimulation input terminal 14 transmitting electrical stimuli to the deposited graphene layer 12.

The base substrate 11 may physically implement the basic structure of the culture substrate 10 while forming mechanical durability.

The base substrate 11 may be at least one of ceramic material, glass, polymer or plastics in which copolymer is polymerized and cured.

For instance, the base substrate 11 may include polystyrene (PS).

However, the constituent material of the base substrate 11 is not limited thereto, but may be changed as long as those skilled in the art may easily change a design within an appropriate range of cell culture.

As described above, in the cell culture vessel 100 according to an embodiment of the present disclosure, the culture substrate 10 may dispose the deposited graphene layer 12 on the base substrate 11, and an oxygen functional group may be formed on a surface of the deposited graphene layer 12.

Specifically, by disposing the deposited graphene layer 12 on the base substrate 11, cell culture uniformity can be improved, contamination can be minimized, and easy long-term storage is possible.

Since the graphene of the deposited graphene layer 12 is an inorganic material composed of carbon and has a regular crystal structure, cell culture uniformity can be improved, the possibility of contamination is low, and storage is easy.

That is, compared to a conventional case in which a substrate is treated with protein coating (e.g., extracellular matrix), the crystal structure is regular, thus enabling uniform cell culture. Furthermore, since protein or the like is easily denatured by changes in external environment (temperature, moisture, and bacteria), the cell culture vessel 100 according to an embodiment of the present disclosure can facilitate long-term storage while minimizing contamination.

Furthermore, graphene may have a similar function to extracellular matrix protein, which is essential for adsorbing cells and supplying nutrients in the cell culture, and thereby may have high binding compatibility with cells.

Moreover, a surface of the deposited graphene layer 12 included in the cell culture vessel 100 according to an embodiment of the present disclosure may be modified to form the oxygen functional group on the surface of the deposited graphene layer 12.

Specifically, by connecting and adding oxygen to carbon forming the deposited graphene layer 12 or substituting oxygen for carbon, the oxygen functional group may be formed on the surface of the deposited graphene layer 12.

The oxygen functional group formed on the deposited graphene layer 12 may include at least one of an epoxy group (C—O—C), a hydroxyl group (—OH), a carboxyl group (—COOH), or a carbonyl group (C═O).

The cell culture vessel 100 according to an embodiment of the present disclosure may improve the adhesiveness or adsorption of cells to be cultured, by forming the oxygen functional group on the surface of the deposited graphene layer 12.

Specifically, various functional groups having high binding properties with the oxygen functional group may be disposed in the cells to be cultured. These oxygen functional groups serve to increase hydrophilicity. This facilitates a process in which cells in culture fluid are attached to the substrate in an initial culture stage.

Such an improvement on initial adhesion properties can improve cell culture efficiency.

The deposited graphene layer 12 included in the cell culture vessel 100 according to an embodiment of the present disclosure can effectively culture cells by the oxygen functional group formed on the surface.

Specifically, the delivery of nutrients can be improved by the oxygen functional group formed on the surface of the deposited graphene layer 12, thus allowing cells to be effectively cultured.

The oxygen functional group is electrically negatively charged, whereas amino acid which is a nutrient required for the cell culture is electrically positively charged. The amino acid which is the nutrient can be easily adsorbed with the oxygen functional group formed on the surface of the deposited graphene layer 12.

Therefore, the supply of nutrients to the cultured cell can be increased, thereby maximizing the cell culture and differentiation efficiency.

The deposited graphene layer 12 included in the cell culture vessel 100 according to an embodiment of the present disclosure may be a single layer or a multiple layer in which a plurality of layers are stacked. The number of layers in the deposited graphene layer 12 may be 1 to 5.

Herein, the deposited graphene layer 12 is the concept which is contrary to flake-type graphene formed by pulverizing graphite. Unlike the flake-type graphene which is formed by stacking several tens or hundreds of graphene layers, the deposited graphene layer 12 may have several graphene layers forming the deposited graphene layer 12, which may be significantly less than that of the flake-type graphene.

In particular, the cell culture vessel 100 according to an embodiment of the present disclosure may include the multi-layered deposited graphene layer 12, thereby improving cell culture efficiency.

Specifically, as described above, the oxygen functional group included in the deposited graphene layer 12 may be formed by connecting and adding oxygen to carbon constituting graphene or substituting oxygen for carbon. Therefore, when the deposited graphene layer 12 is formed of multiple layers, the oxygen functional group may be additionally formed for each layer. Thus, as the number of spots to which cells to be cultured will be adsorbed increases, the cells can be effectively cultured.

In the multi-layered deposited graphene layer 12, the number of oxygen functional groups may increase from the lowest layer to the uppermost layer.

Herein, the lowest layer means a graphene layer closest to the base substrate 11, and the uppermost layer means a graphene layer farthest from the base substrate 11.

Since the oxygen functional group may be formed when the surface of the deposited graphene layer 12 is damaged by oxidation plasma treatment which will be described later and carbon constituting graphene is substituted, the most oxygen functional group may be present in the uppermost layer of the deposited graphene layer 12, which has the biggest damage due to the oxidation plasma treatment, and the oxygen functional group may be decreased toward the lowest layer.

However, the deposited graphene layer 12 of the cell culture vessel 100 according to an embodiment of the present disclosure controls the number of graphene layers forming the deposited graphene layer 12 within 5 layers. This prevents the oxidation plasma treatment from being adversely affected due to too many graphene layers, thus allowing the oxygen functional group to be formed even in the graphene layer adjacent to the lowest layer.

The cell culture vessel 100 according to an embodiment of the present disclosure can effectively transmit electrical stimuli through the electrostimulation input terminal 14 to the cells which are cultured while being adhered or adsorbed to the deposited graphene layer 12 of the culture substrate 10, thus improving cell culture efficiency.

Turning back to FIGS. 1 to 3, the electrical stimuli transmitted to the cultured cells may be transmitted from a power supply device 50 to an electrode 40, and then the electrical stimuli transmitted to the electrode 40 may be transmitted to the deposited graphene layer 12 and finally transmitted to the cultured cells.

Specifically, the power supply device 50 may transmit electrical stimuli through the working electrode 41 and the counter electrode 42 to the deposited graphene layer 12.

The working electrode 41 and the counter electrode 42 may be electrically connected to the power supply device 50 via a separate wire or the like. However, a connection structure of the electrode 40 and the power supply device 50 is not limited to that shown in the drawings and the above description, but may be easily changed in design by those skilled in the art.

Furthermore, herein, the working electrode 41 and the counter electrode 42 mean electrodes having polarities which are electrically opposite to each other, and the working electrode 41 or the counter electrode 42 does not mean a specific polarity.

Subsequently, the electrostimulation input terminal 14 may be directly coupled to the deposited graphene layer 12, and simultaneously the electrostimulation input terminal 14 may be directly coupled to the working electrode 41.

That is, since the electrostimulation input terminal 14 is directly coupled to the working electrode 41 and simultaneously is directly coupled to the deposited graphene layer 12, the electrical stimuli transmitted from the working electrode 41 can be effectively applied to the deposited graphene layer 12, without the necessity of providing a separate electrical stimulation mediator with high resistance.

Since the deposited graphene layer 12 is a material with excellent electric conductivity, the electrical stimuli applied to the deposited graphene layer 12 can be effectively transmitted to cells which are cultured while being adhered and adsorbed to the surface of the deposited graphene layer 12, and consequently a cell culture capability can be improved.

Meanwhile, the coupling structure of the working electrode 41 and the deposited graphene layer 12 may be different from that of the counter electrode 42 and the deposited graphene layer.

Specifically, as described above, the working electrode 41 may be directly coupled to the culture substrate 10 through the electrostimulation input terminal 14, but the counter electrode 42 may be located to be spaced apart from the culture substrate 10, for example, be located in the culture fluid held in the cell culture vessel 100.

That is, in the cell culture vessel 100 according to an embodiment of the present disclosure, selectively, the working electrode 41 may directly apply electrical stimuli to the culture substrate 10 through the electrostimulation input terminal 14, but the counter electrode 42 may be disposed to be spaced apart from the culture substrate 10 with the culture fluid having relatively significant electrical resistance therebetween.

Moreover, at least one of the working electrode 41 or the counter electrode 42 may include at least one of Pt, Au, Ag, Ti, or stainless steel to efficiently transmit electrical stimuli.

However, the constituent materials of the working electrode 41 and the counter electrode 42 are not limited thereto, but will include a material which may be easily changed in design by those skilled in the art.

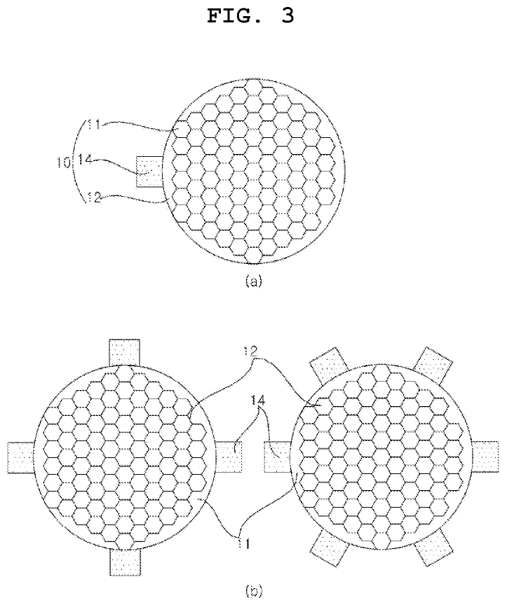

FIG. 3 illustrates different examples of the culture substrate 10 according to an embodiment of the present disclosure. The electrostimulation input terminal 14 may be disposed on the culture substrate 10 in various forms.

Specifically, FIG. 3(a) is a plan view showing a case including a single electrostimulation input terminal 14, and FIG. 3(b) is a plan view showing a case including a plurality of electrostimulation input terminals 14.

Specifically, referring to FIG. 3(a), the electrostimulation input terminal 14 may be disposed on an edge of the deposited graphene layer 12 or the base substrate 11. Further, even if the electrostimulation input terminal is disposed on the edge of the base substrate 11, the electrostimulation input terminal may be electrically directly connected to the deposited graphene layer 12.

Therefore, the electrical stimuli applied from the edge of the deposited graphene layer 12 or the base substrate 11 to the electrostimulation input terminal 14 may be diffused and transmitted from a portion close to the electrostimulation input terminal 14.

Meanwhile, the deposited graphene layer 12 which is distant from the electrostimulation input terminal 14 may be reduced in electrical stimuli due to self-resistance. It is preferable that the electrical stimuli applied to the cells to be cultured are uniform so as to effectively culture the cells.

Therefore, referring to FIG. 3(b), the cell culture vessel 100 according to an embodiment of the present disclosure may be provided with a plurality of electrostimulation input terminals 14. The electrostimulation input terminals 14 may be disposed on the edge of the deposited graphene layer 12 or the base substrate 11 to be spaced apart from each other.

As a result, the electrical stimuli transmitted by the plurality of electrostimulation input terminals 14 which are uniformly disposed on the edge of the culture substrate 10 may be transmitted to cells which are uniformly cultured in the entire area of the culture substrate 10.

When the plurality of electrostimulation input terminals 14 are provided, a plurality of working electrodes 41 should be provided to correspond to the number of the electrostimulation input terminals, unlike that shown in the drawing.

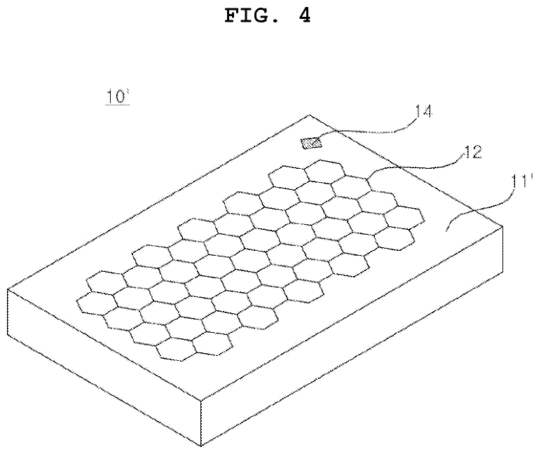

Next, a cell culture vessel 100 according to another embodiment of the present disclosure will be described with reference to FIG. 4. The same description may be applied to the remaining components of a culture substrate 10′ of the cell culture vessel 100 according to an embodiment of the present disclosure except for structures and components different those of the above-described culture substrate 10.

Thus, differences will be mainly described below.

In the culture substrate 10′ of the cell culture vessel 100 according to another embodiment of the present disclosure, a base substrate 11′ may include a conductive metal layer, and specifically, may include at least one of ITO, Ag, Au, or Cu.

The conductive metal layer of the base substrate 11′ is a material having significantly excellent electric conductivity compared to the deposited graphene layer 12, is not limited to the above-described kind, and may be easily changed in design by those skilled in the art.

In the culture substrate 10′, the deposited graphene layer 12 may be directly disposed on the base substrate 11′ made of the conductive metal layer, and the electrostimulation input terminal 14 may be directly coupled to the base substrate 11′.

That is, in the culture substrate 10′, the electrostimulation input terminal 14 does not necessarily have to be directly coupled to the deposited graphene layer 12, or the deposited graphene layer 12 and the electrostimulation input terminal 14 do not have to be separately connected in the culture substrate 10′, as long as the electrostimulation input terminal 14 and the deposited graphene layer 12 are disposed on the culture substrate 10′ including the conductive metal layer.

In the culture substrate 10′ according to an embodiment of the present disclosure, the base substrate 11′ includes the conductive metal layer having the excellent electric conductivity, so that the electrical stimuli can be more effectively applied to the entire deposited graphene layer 12.

Specifically, since the base substrate 11′ includes the conductive metal layer having very good electric conductivity, the electrical stimuli may be effectively transmitted and applied even to a portion of the deposited graphene layer 12 which is distant from the electrostimulation input terminal 14 via the base substrate 11′.

Moreover, since the base substrate 11′ itself serves as a medium for transmitting the electrical stimuli, the electrostimulation input terminal 14 does not necessarily need to be located on the same surface as the deposited graphene layer 12.

Further, the position of the electrostimulation input terminal 14 is not limited as long as the electrostimulation input terminal is located on a surface of the base substrate 11′. Thus, the shape and structure of the working electrode 41 are not limited to those which are already disclosed in the above description and drawings.

For example, the electrostimulation input terminal 14 may be located on the bottom of the base substrate 11′ on which the deposited graphene layer 12 is disposed, and the base substrate 11′ including the conductive metal layer itself becomes the working electrode 41, so that a wire connected to the power supply device 50 may be directly coupled and electrically connected to the electrostimulation input terminal 14.

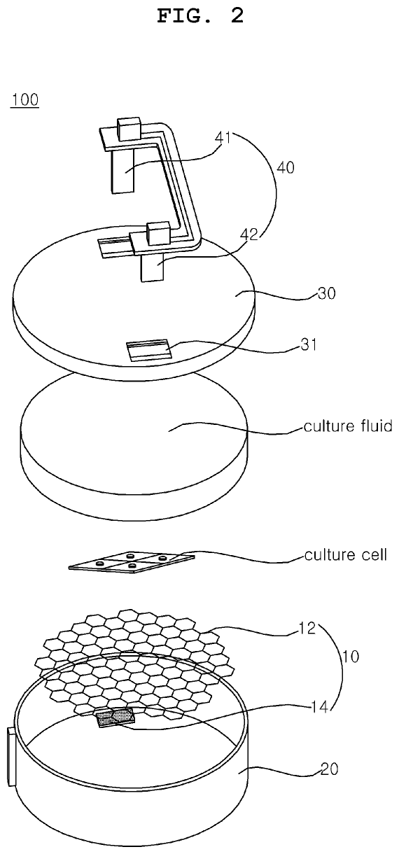

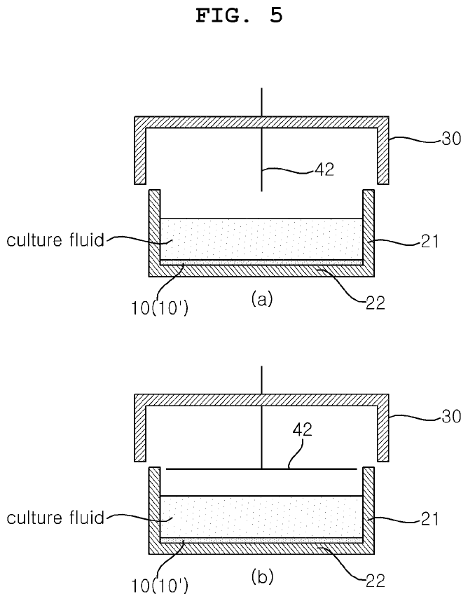

Turning back to FIGS. 2 and 5, additional components of the cell culture vessel 100 according to an embodiment of the present disclosure will be described.

The cell culture vessel 100 may include a culture substrate case 20 including a case bottom 22 on which the culture substrate 10 or 10′ is placed, and an outer wall 21 surrounding an edge of the case bottom 22.

The cell culture vessel may further include a culture substrate case cover 30 covering an upper opening of the culture substrate case 20.

The culture substrate case 20 and the culture substrate case cover 30 coupled to each other may be divided so that an inner space is separated from the outside, thus preventing external bacteria from entering and preventing the culture cells from being contaminated.

Of course, the culture substrate case cover 30 may be provided with at least one electrode insertion hole 31 through which at least one of the working electrode 41 or the counter electrode 42 passes.

The electrode insertion hole 31 is an opening, which may be shielded by inserting the working electrode 41 or the counter electrode 42 therein.

Moreover, the number and shape of the electrode insertion hole 31 are not limited to those shown in the drawing, but may be easily changed in design by those skilled in the art within a range corresponding to the number, arrangement, and shape of each of the working electrode 41 and the counter electrode 42.

The cell culture vessel 100 according to an embodiment of the present disclosure can effectively apply uniform electrical stimuli to the culture substrate 10 or 10′ by controlling the shape of the counter electrode 42 as well as the culture substrate 10 or 10′.

Specifically, the counter electrode 42 may have a plate shape parallel to the culture substrate 10 or 10′, or a linear shape intersecting the culture substrate 10 or 10′.

Referring to FIG. 5(a), the counter electrode 42 may be linearly located to intersect, for example, be perpendicular to the culture substrate 10 or 10′, and simultaneously be located at the center of the culture substrate 10 or 10′, thus preventing a portion of the culture substrate 10 or 10′ from being too far away from the counter electrode 42 and thereby preventing electrical stimuli from becoming significantly non-uniform.

Moreover, referring to FIG. 5(b), the counter electrode 42 may have the plate shape parallel to the culture substrate 10 or 10′, so that a distance from each portion of the deposited graphene layer 12 to the counter electrode 42 is constant and simultaneously shortened, and thereby it is possible to effectively and uniformly apply electrical stimuli to the entire culture substrate 10 or 10′.

Although the present disclosure was described with reference to specific embodiments shown in the drawings, it is apparent to those skilled in the art that the present disclosure may be changed and modified in various ways without departing from the scope of the present disclosure, which is described in the following claims.

What is claimed is:

1. A cell culture vessel comprising a culture substrate comprising:
a base substrate;
at least one deposited graphene layer disposed on the base substrate; and
at least one electrostimulation input terminal coupled to a working electrode and configured to transmit electrical stimuli to the deposited graphene layer.

2. The cell culture vessel of claim 1, wherein the electrostimulation input terminal is directly coupled to the deposited graphene layer.

3. The cell culture vessel of claim 2, wherein a plurality of electrostimulation input terminals are disposed on an edge of the deposited graphene layer to be spaced apart from each other.

4. The cell culture vessel of claim 1, wherein the base substrate comprises polystyrene (PS).

5. The cell culture vessel of claim 1, wherein the base substrate comprises a conductive metal layer, and the conductive metal layer comprises at least one of ITO, Ag, Au, or Cu.

6. The cell culture vessel of claim 5, wherein the deposited graphene layer is directly disposed on the base substrate, and the electrostimulation input terminal is directly coupled to the base substrate.

7. The cell culture vessel of claim 5, further comprising:
the working electrode transmitting electrical stimuli to the deposited graphene layer, and a counter electrode electrically opposite to the working electrode,
wherein the working electrode is directly coupled to the culture substrate, and the counter electrode is located to be spaced apart from the culture substrate.

8. The cell culture vessel of claim 7, wherein the counter electrode has a plate shape parallel to the culture substrate or a linear shape intersecting the culture substrate.

9. The cell culture vessel of claim 7, further comprising:
a culture substrate case comprising a case bottom on which the culture substrate is placed, and an outer wall surrounding an edge of the case bottom; and
a culture substrate case cover covering an upper opening of the culture substrate case,
wherein the culture substrate case cover comprises at least one electrode insertion hole through which at least one of the working electrode or the counter electrode passes.

10. The cell culture vessel of claim 7, wherein the working electrode and the counter electrode are connected to a power supply.

11. The cell culture vessel of claim 7, wherein at least one of the working electrode or the counter electrode comprises at least one of Pt, Au, Ag, Ti, or stainless steel.

12. The cell culture vessel of claim 1, wherein the deposited graphene layer is a single layer or a multiple layer in which a plurality of layers are stacked, and
the number of layers in the deposited graphene layer is 1 to 5.

13. The cell culture vessel of claim 1, wherein an oxygen functional group is formed on a surface of the deposited graphene layer.
